(19) United States
(12) Patent Application Publication
(10) Pub. No.: US 2008/0033491 A1
(43) Pub. Date: Feb. 7, 2008

(54) Implantable Device and Method for Transvascular Neuromodulation

(76) Inventor: Stephen M. Zappala, Andover, MA (US)

Correspondence Address: MIRICK, O'CONNELL, DEMALLIE & LOUGEE, LLP, 1700 WEST PARK DRIVE, WESTBOROUGH, MA, 01581, US

(21) Appl. No.: 11/753,679

(22) Filed: May 25, 2007

Related U.S. Application Data
(63) Continuation-in-part of application No. 11/342,113, filed on Jan. 28, 2006.
Application No. 11/342,113 is a division of application No. 10/005,390, filed on Dec. 3, 2001, now Pat. No. 6,993,390.

(51) Int. Cl. A61N 1/00

(52) U.S. Cl. 607/2

(57) ABSTRACT

A method for using an implantable device to help manage a patient's condition. The device includes a power source member that provides power to a pulse-generating member. An electrode member has its proximal end electrically connected to the pulse-generating member, and a distal end with one or more electrodes, the distal end being located intravenously such that the electrode(s) are proximate an area such as an organ to be treated by the electrical pulses.

### CROSS-REFERENCE TO RELATED APPLICATION

This application is a continuation in part of application Ser. No. 11/342,113, filed Jan. 28, 2006, which is itself a divisional of application Ser. No. 10/005,390, filed on Dec. 3, 2001. Priority of both applications is claimed. Both applications are incorporated herein by reference.

### FIELD OF THE INVENTION

The invention relates to a method and implantable device for transvascular neuromodulation.

### BACKGROUND OF THE INVENTION

Conventional therapies in the management of chronic medical disease processes have historically (typically) required systemic medical therapies, which are non-specific and delayed in their onset of clinical action. Moreover, such medical therapies have been documented to have significant morbidities and decreased clinical compliance.

One example of such is erectile dysfunction (ED), which is the persistent inability to attain and maintain penile erection sufficient for vaginal intercourse; ED is a major health issue among males and especially among the aging male population. The etiology of ED is functional and includes vasculogenic, neurogenic, endocrinologic and psychogenic, and is usually associated with vascular disease, endocrinopathy or a neural injury to the central or peripheral nervous system. Management options for ED depend on the cause of the dysfunction and include medical and surgical therapies and vacuum erection devices, each with their own limitations and complications.

Medical therapies include the oral, transcutaneous (penile injection) and transurethral (e.g. MUSE System) routes of delivery of various pharmacologic agents. See, for example, U.S. Pat. No. 5,916,569 to Spencer et al., U.S. Pat. No. 5,925,629 to Place, and U.S. Pat. No. 6,156,753 to Doherty, Jr. et al. However, many men are not suitable candidates for oral agents such as sildenafil (Viagra; Pfizer, N.Y.), a phosphodiesterase inhibitor, because of potential life threatening interactions with cardiac medications such as nitrates.

Penile (intracavemosal) injection therapy with vasodilator agents such as prostaglandin E₁, papaverine, nitric oxide, phentolamine, apomorphine, or vasoactive intestinal peptide (VIP) is a well-accepted method. The technique however requires instruction to anxious patients with careful attention to the dose, injection sites, and the amount of the agent. Many patients withdraw from intracavernosal injection therapy because of the anxiety associated with self-injection, recurrent cutaneous ecchymoses, painful injections, or associated corporal fibrosis (Peyronie's Disease). Moreover, patients are uncomfortable when they travel through public airports or to foreign countries with syringes and medications. These limitations, associated with the complete loss of spontaneity, represent the primary reasons for discontinuation in an otherwise successful pharmacologic erection program.

Surgically invasive procedures have been reserved for those men who fail conservative therapies; these options include revascularization procedures, penile prostheses and cavernous nerve stimulation devices, e.g. U.S. Pat. No. 5,938,584 to Ardito et al. and U.S. Pat. No. 6,169,924 B1 to Meloy et al. Penile prostheses are generally last resort because implantation results in irreparable damage to the cavernosal tissue. Agents and devices specifically designed to stimulate the NVB of the phallus have not previously been successful because of the size of the NVB, sensitivity of the NVB to neural fibrosis, and extensive distal, neural damage resulting from surgical procedures such as a radical retropubic prostatectomy.

### SUMMARY OF THE INVENTION

It is therefore a primary object of this invention to provide an implantable neuromodulating device and method using such a device, for managing chronic medical diseases.

The invention can be used for at least the following purposes:

Dermatology/Cutaneous:

Thermal injuries

Raynaud's disease

Vasculitis

HEENT:

Retinal spasm

Meniere's disease.

Carotid Stenosis

Cerebrovascular disease

sleep apnea

chronic sinusitis

Pulmonary/Thoracic:

Pulmonary hypertension

Hiccups

Asthma, COPD

Cardiac:

Arrhythmias

post infarction hyperperfusion

angioplasty neovascularity

CABG—revascularization

Gastroenterology:

Esophageal spasm

Gastro-esophageal Reflux Disease GERD

Hypomotility, hypermobility

Splanchnic, mesenteric angina

chronic constipation

elimination syndrome

dumping syndrome

Neurology:

Vertebral, subdlavian, and carotid steal syndromes

Post Cerebrovascular Injury

Enhancement of neovascularity

Motion Disorders
Hyperactivity
Hypoactivity
Spasticity
Parkinson's Disease
Huntington's Chorea

Alzheimer's Disease

Multiple Sclerosis

Spinal Cord Injury

Seizures

Urology:

Male and Female Sexual Dysfunction

Urinary Incontinence

Atonic and Neuropathic Bladder

Pelvic Pain Syndrome/Interstitial Cystitis

Ejaculatory dysfunction

Retrograde ejaculation

Anorgasmia

Rheumatology:

Inflammatory syndromes

vasculitis

Endocrinology:

Diabetes: Splenic vein catheterization to elicit pancreatic islet stimulation of autologous Insulin or Glucagon

Thyroid

Adrenal

Vascular:

Neuromodulation of vascular disease

“RF sympathectomy”, central and peripheral

Neovascular collateral stimulation

Renovascular Hypertension

Essential Hypertension

Orthopedic:

bone growth stimulation to treat and prevent osteopenia, osteoporosis

bone deposition for fracture healing

Plastic/Reconstructive Surgery:

improved graft/flap survival

Oncology:

Primary: release of tumor mediators and cytokines

Secondary: Improved vascular supply in conjunction with chemotherapy

Veterinary:

Lactation

Menstrual Cycle regulation

Seminal emission

The preferred embodiment of the implantable device of the invention generally comprises at least one power source member that is adapted to be implanted in the patient; at least one pulse generating member that is adapted to be implanted in the patient; and at least one electrode that is adapted to be implanted in the patient, typically intravenously. The electrode is connected to the pulse generating member, and is adapted to electrically stimulate tissue such as a neurovascular bundle, or an organ. Stimulation is accomplished either under patient control, or automatically in response to one or more detected biological conditions.

The device may further comprise an elongated lead, to which the electrode is fixed, that connects the electrode to the pulse-generating member. The lead preferably has an outside diameter of about 2 mm or less, to which the electrode is attached and may comprise at least one extension cable having a length sufficient to connect the electrode to the pulse generating member. There may be a means for remotely activating the power source member and pulse-generating member. The power source member preferably comprises a high impedance battery. The pulse-generating member preferably emits low amplitude, high frequency pulses. The power source member and the pulse-generating member may be deactivated automatically when a predetermined electrical potential is reached. The power source member and pulse-generating member of the invention may be deactivated automatically after a predetermined temporal period has passed. The two are preferably housed together within a titanium shell that is adapted to be implanted in a subcutaneous location in the patient. The pulse-generating member may emit electrical pulses of about 10 to 40 Hz and 1 to 5.5 V. The electrode member is preferably provided with one or more electrodes that comprise an indifferent material.

This invention features a method for managing a patient's condition, comprising the steps of: providing an implantable delivery device, comprising at least one power source member, at least one pulse-generating member, and at least one electrode member having an active portion with one or more electrodes, the electrode member electrically connected to the power source member and/or the pulse generator, and the electrodes adapted to provide electrical stimulation. The device is surgically implanted in the patient such that the power source and pulse generator are in the body and the active portion of the electrode member is located in one or more blood vessels. The pulse generator is selectively activated, to generate electrical pulses that are provided to one or more of the electrodes, to electrically stimulate the patient's tissue proximate the electrodes. The patient may be human or non-human.

The power source member and the pulse-generating member may be housed in a biocompatible shell that is adapted to be implanted in the patient. The pulse-generating member may be adapted to generate pulses of about 10 to about 40 Hz and about 1 to about 5.5 V. The pulse-generating member may emit low amplitude, high frequency pulses.

The power source member and the pulse-generating member may be adapted to be deactivated automatically after a predetermined temporal period has passed. The device may further comprise an elongated lead, to which the electrode member is fixed, that connects the electrode member to the power source member and pulse-generating member. The power source member may comprise a high impedance battery. The device may further comprise a lead with an outside diameter of about 2 mm or less, to which the electrode member is attached and comprises at least one extension cable having a length sufficient to connect the electrode member to the power source member and the pulse generating member. The power source member and the pulse generating member may be adapted to be deactivated automatically when a predetermined electrical potential is reached.

The implantable delivery device may further comprise an in vivo biological monitor that is electrically coupled to the pulse-generating member. The step of selectively activating the pulse generator may be responsive to the biological monitor, such that the pulse generator is activated in response to an internal condition sensed by the biological monitor. The pulse generator may also be deactivated in response to an internal condition sensed by the biological monitor.

### BRIEF DESCRIPTION OF THE DRAWINGS

Other objects, features and advantages will occur to those skilled in the art from the following description of the preferred embodiments and the accompanying drawings in which:

FIG. 1 is a side view of the electrical leads of an embodiment of the device of the invention;

FIG. 2 is a cross-sectional side view of the patient's neurovascular supply of the phallus within which the embodiment shown in FIG. 1 is adapted to be implanted according to the method of the invention;

FIG. 3 is a perspective view of an embodiment of the device of the invention in a percutaneous position;

FIG. 4 is a perspective view of an embodiment of the device of the invention implanted in a surgical position; and

FIGS. 5-14 are schematic views of the inventive device implanted for use in: Peripheral Vascular Disease (FIG. 5); Veterinary (FIG. 6); Renovascular Disease (FIG. 7); Diabetes Mellitus (FIG. 8); Carotid Disease (FIG. 9); Urogynecology (FIG. 10); Erectile Dysfunction (FIG. 11); Neurology (FIG. 12); Asthma, Pulmonary Hypertension, Hypoxemia and Bronchodilation (FIG. 13); and Post Prandial glucose regulation of type II Diabetes Mellitus (FIG. 14), respectively.

### DETAILED DESCRIPTION OF THE PREFERRED EMBODIMENTS

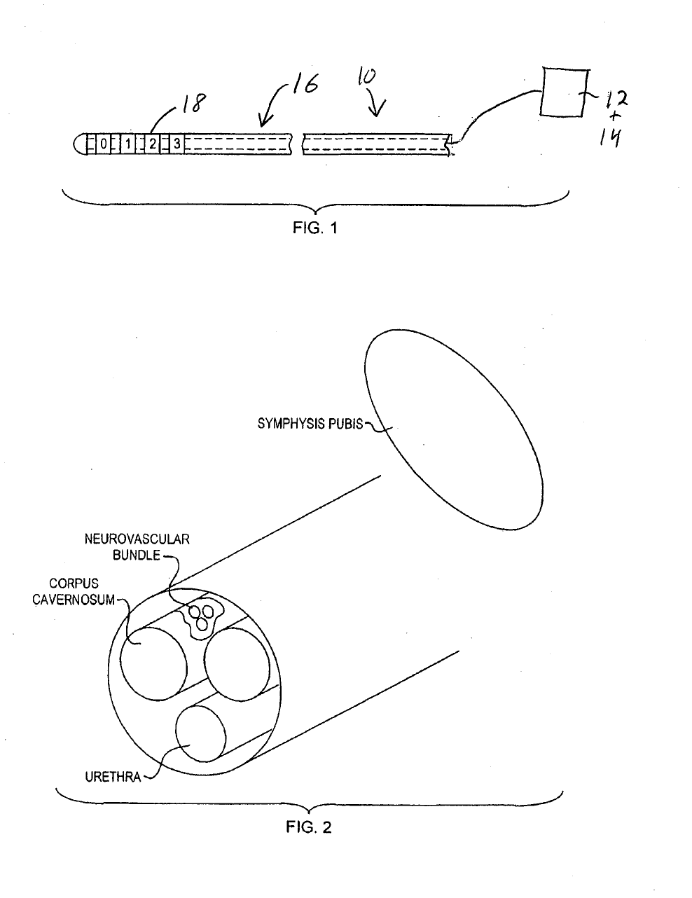

One embodiment of the inventive device, shown in FIGS. 1-4, is an implantable, transvenous neural stimulator that applies a stimulating, low electrical voltage to the NVB of the phallus as the primary or adjunctive therapy of erectile dysfunction. The device is activated by an external signaling source and will deactivate spontaneously after a temporal period or when a predetermined electrical potential is reached. The device is preferably surgically implanted into the hypogastric, internal iliac, pudendal, or the dorsal vein of the phallus with the generator and the battery positioned into a subcutaneous pouch of the lower abdominal wall. A test or simulation procedure can be performed prior to permanent implantation of the device. The device is multiprogrammable from the external source.

The neurovascular anatomy of the phallus is relatively constant with the neural tissue routinely identified and located within the intercavernosal space. The NVB contains both neural and vascular structures (arteries, veins) and its course runs parallel to the cavernosal bodies. Both structures are subcutaneous within the phallus (FIG. 2) and proximally diverge at the level of the membranous urethra. The dorsal vein is located anterior to the membranous urethra while the neural bundles diverge over the dorsolateral aspect of the prostate. NVB stimulation is associated with relaxation of the corpus cavernosal smooth muscle, tunica albuginea, and vascular dilation via the release of vasoactive transmitters such as vasoactive intestinal peptide and nitric oxide.

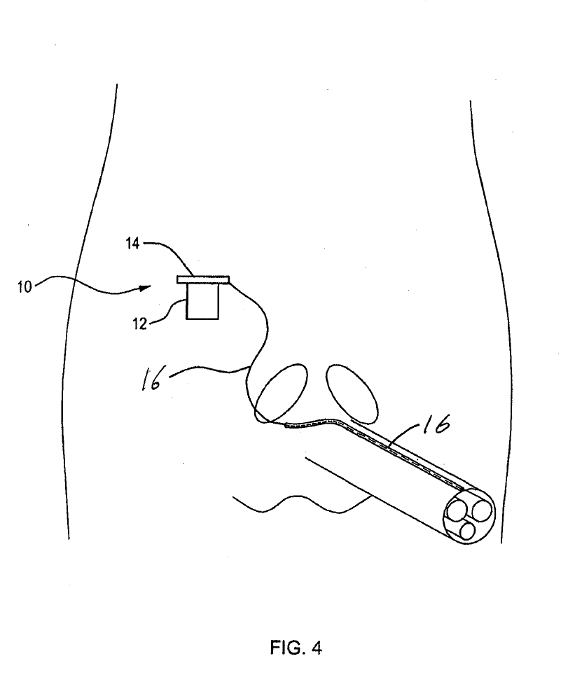

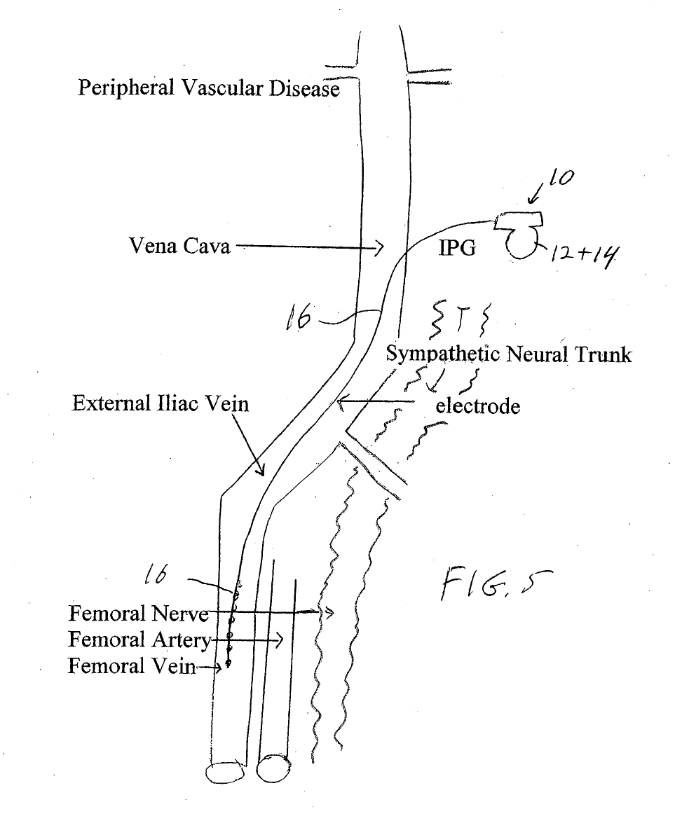

The dorsal vein neuromodulator (DVN) of the invention, generally shown and referred to in FIG. 4 as device 10, is a surgically implanted device which generally comprises three primary components: high impedance battery/power source 12, pulse generator 14 (low amplitude, high frequency), and electrode member 16 which preferably comprises at least four electrodes 18 (labeled 0, 1, 2 and 3—see FIG. 1). Device 10 is preferably single use and latex free. Electrode member 16 is preferably a small calibre lead with an outside diameter of about 2 mm or less. Optimal lead placement to the phallic NVB via the dorsal vein is critical to the operation and success of the device. An example of electrode member 16 (and extension lead 17 when used) is shown in FIG. 1. The battery and pulse generator are preferably housed together in a biocompatible material and further encapsulated within a thin-wall titanium shell that is surgically implanted in its entirety within a subcutaneous pocket in the lower abdominal wall. The electrode member, which may be unipolar and/or bipolar, is coated with an insulating material such as silicone or polyurethane. The indifferent “pacemaker” tips of the electrodes 18 are preferably uninsulated stainless steel NP35.

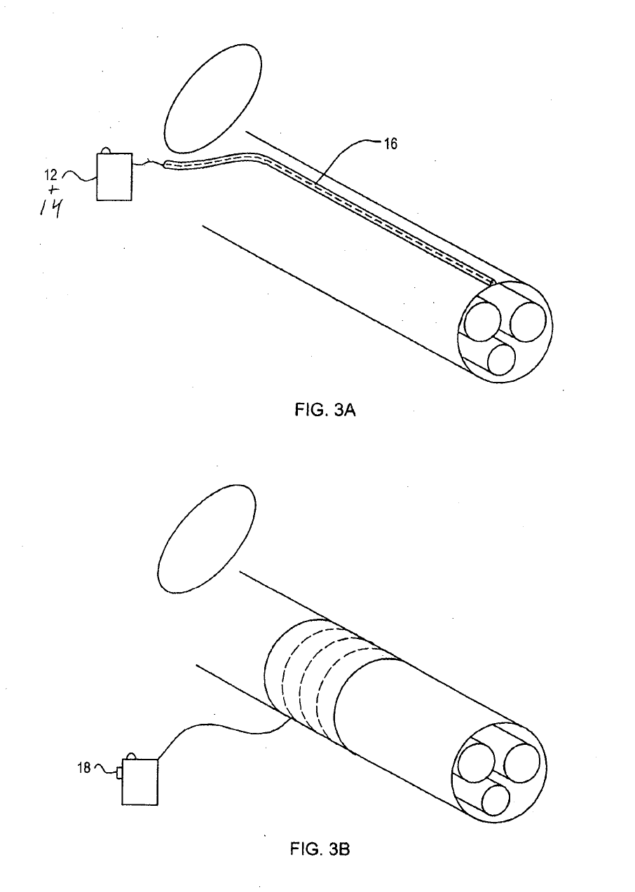

Before permanently implanting the device in a patient, a simulation should be performed to verify the device's potential effectiveness for that particular patient. The primary site for simulation is either transcutaneous (external, prior to radical retropubic or pelvic surgery) or percutaneous via subcutaneous venous collaterals, as shown in FIGS. 3A and 3B, respectively.

The permanent device is implanted at the suprapubic level of the dorsal vein as the vein traverses the rectus abdominis fascia. The vein is cannulated with an insertion sheath and the stylet is removed. The electrode is inserted through the sheath with fluoroscopic guidance to the periphery of the NVB. Electrode activation is performed intraoperative to guarantee the optimal lead position for transvenous stimulation. The electrode is attached to an extension cable that is connected to the battery/generator. The battery and generator are implanted into a subcutaneous pouch of the lower abdominal wall. The device is connected to an external source during the stimulation to the permanently implanted device.

The patient may selectively regulate the amplitude and rate of stimuli (pulse width) of the stimulation through the external or remote source that utilizes a suitable means for communication such as IR and/or RF transmission. The impedance of the tissue is about 800 to 2000 ohms. To stimulate both the striated and smooth muscle fibers of the phallus, the frequency range of the device should be about 10 to 40 Hz and the stimulating voltage should be programmable from about 1 V to 5.5 V. The exact neural voltage is determined based on the particular situation.

The preferred method of the invention for managing erectile dysfunction begins with the step of providing the implantable delivery device of the invention, generally comprising: at least one power source member that is adapted to be implanted in the patient's lower abdominal wall; at least one pulse generating member that is adapted to be implanted in the patient's lower abdominal wall; and at least one electrode that is adapted to be implanted at the suprapubic level of the patient's neurovascular bundle of the phallus, is connected to said power source member and pulse generator, and is adapted to electrically stimulate the neurovascular bundle upon selective activation by the patient surgically implanting said device so that, at least one of said power source members is implanted in the patient's abdominal wall; at least one of said pulse generating members is implanted in the patient's abdominal wall; at least one of said electrode members is implanted at a suprapubic level of the patient's neurovascular bundle via the dorsal vein of the phallus; activating said power source member to initiate said pulse generator to generate electrical pulses to said electrode and electrically stimulate the patient's neurovascular bundle.

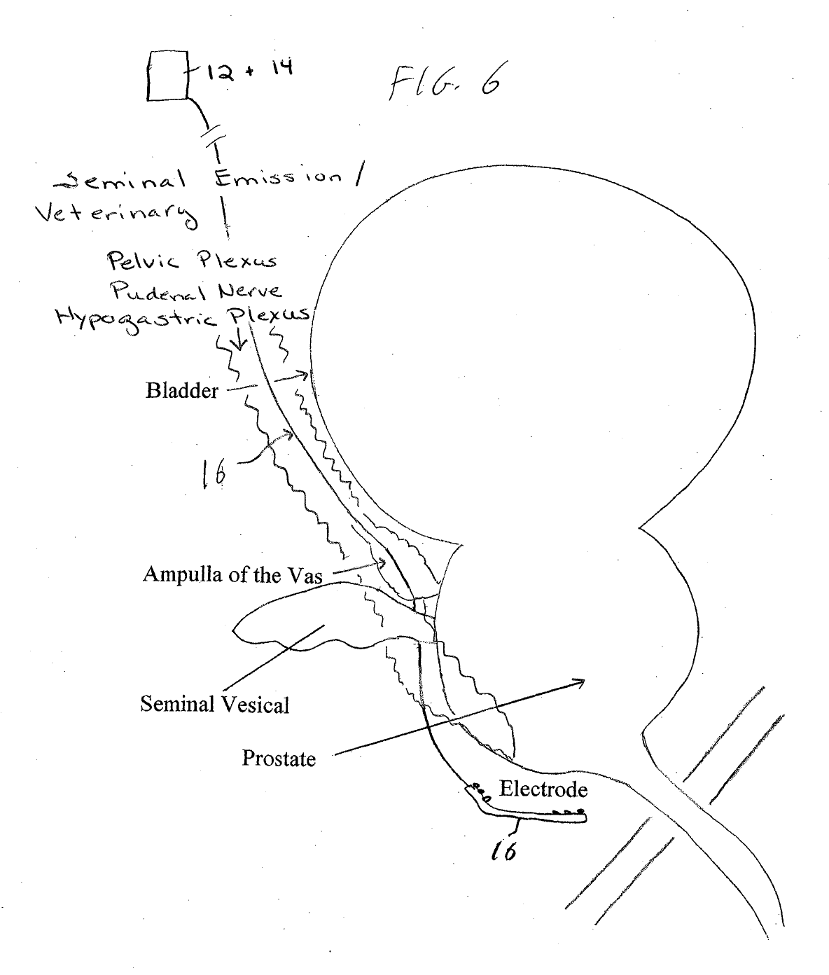

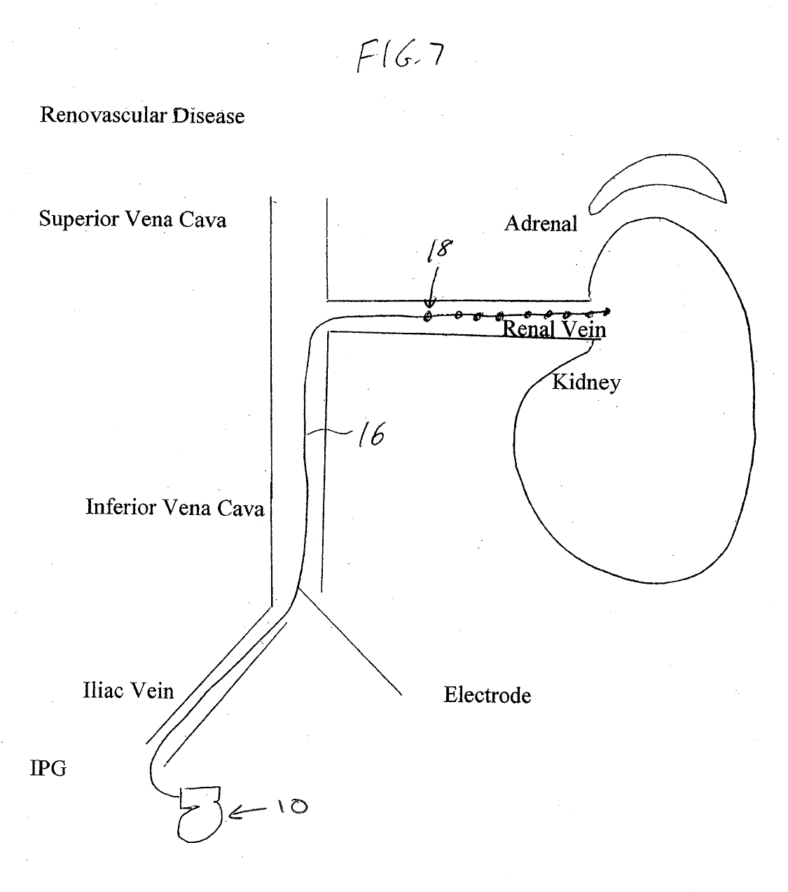

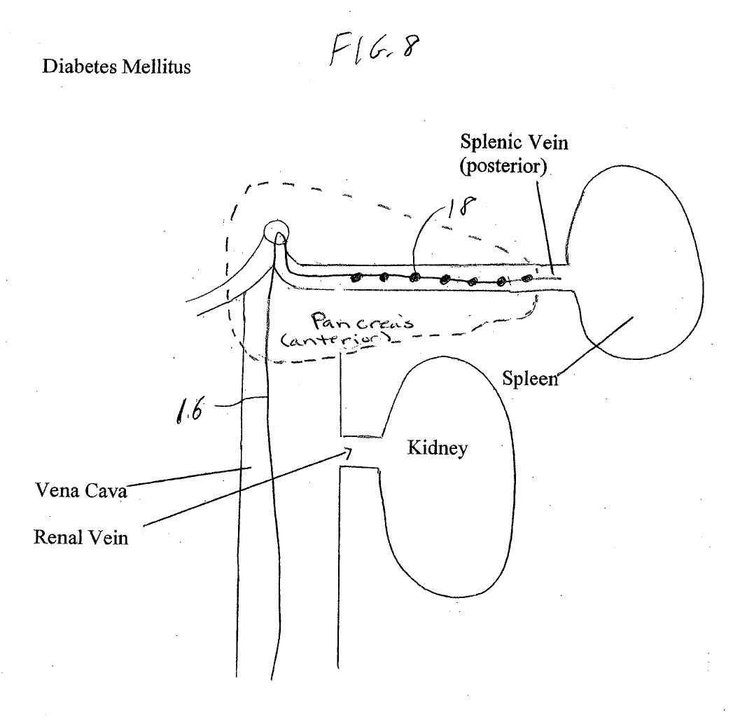

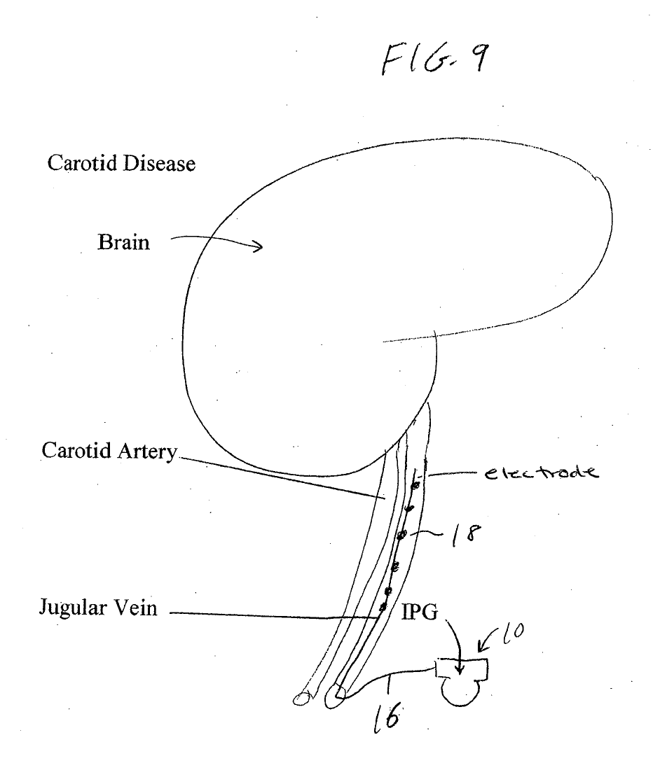

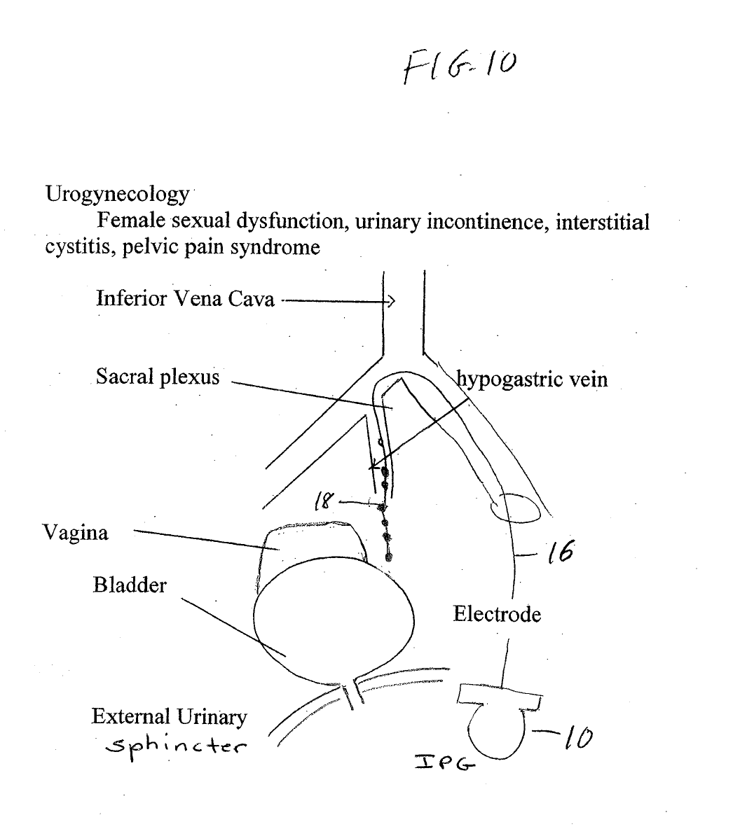

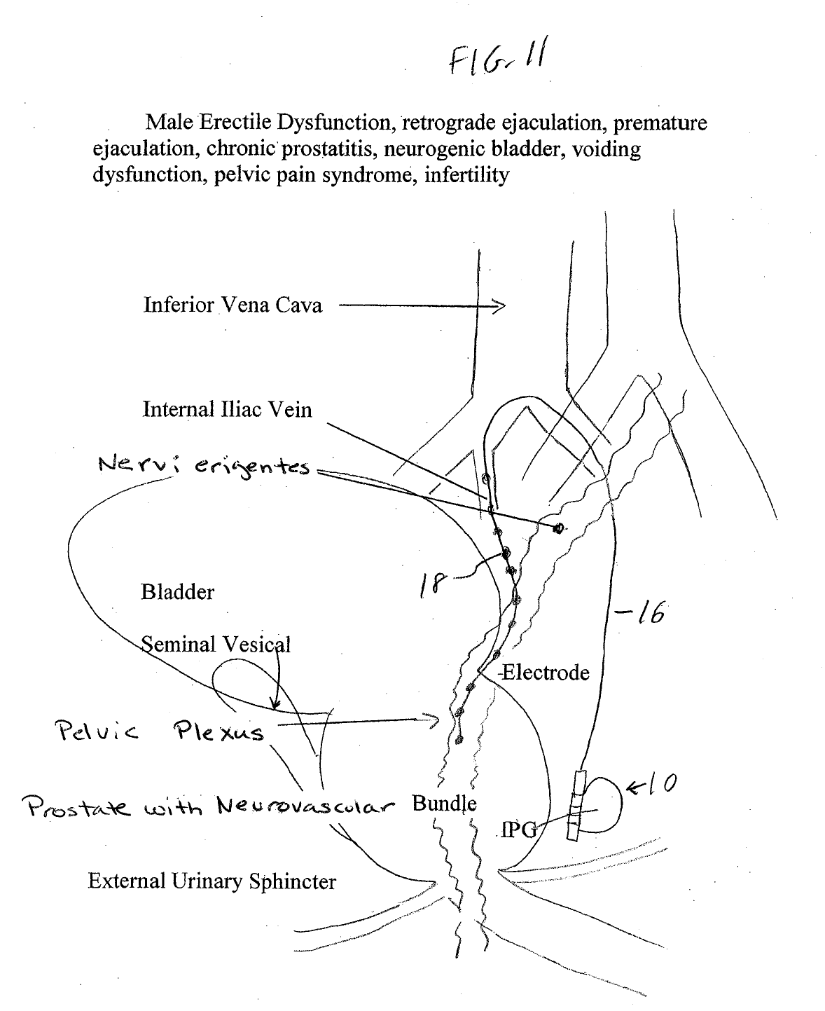

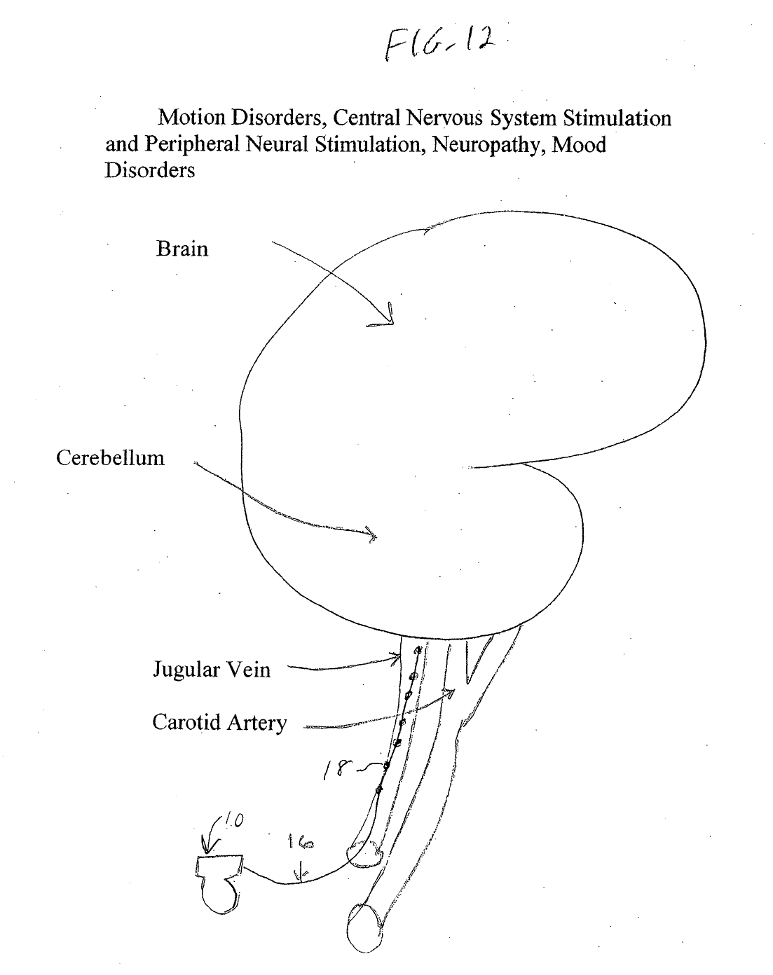

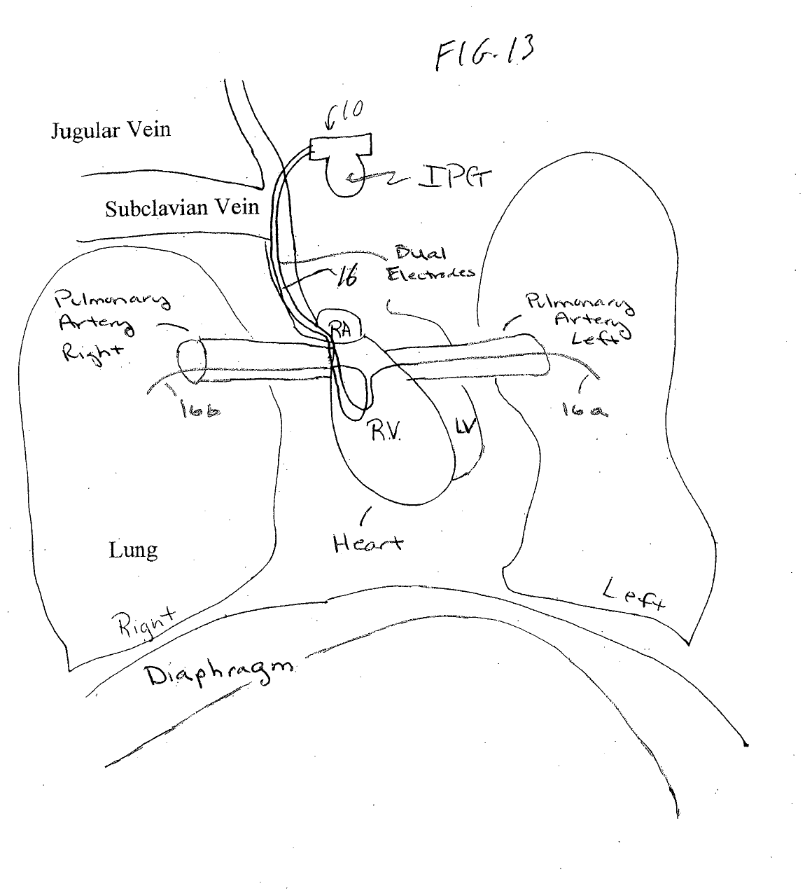

The invention also contemplates use for indications other than ED. Other preferred configurations of the inventive transvascular neuromodulator are depicted in FIGS. 5-14. In the device, the lengths of the inactive (proximal) and active (terminal) ends of the electrode member(s) are tailored to achieve a desired result. The variations in the length of the electrode members are specific to the (1) target organ and the (2) distance from the Implantable Pulse Generator (IPG) to the end terminus. Some organ systems may require multiple electrodes to provide maximum stimulation to the end organ from two or more vascular channels.

The inventive device utilizes the inherent, anatomic, vascular arcades as insulated conduits to deliver the electrode energy to the target site(s). The minimally invasive, transvascular insertion is facilitated by other imaging modalities such as fluoroscopy or laparoscopy or combinations thereof. The activated electrode delivers the desired electrical energy in a unidirectional manner and is completely intraluminal: the intravascular blood is thus transformed into a contiguous medium, which transfers energy along a broad vantage point to the desired target organ or neural pathway.

The IPG electrical voltage and frequency are typically as described above: the frequency is typically from about 10 to about 40 Hz, and the stimulation voltage is typically from about 1 to about 5.5 V.

The IPG is internal, and can be externally activated and/or programmable from an outside source such as a magnet, PDA, or radiofrequency device. The IPG could be in the “OFF” mode and activated for a scheduled, pre-determined temporal period. Additionally, the IPG could be in the “ON” mode and either de-activated or temporarily paused from an external source. The IPG can be activated based on the detection of a predetermined biological parameter such as blood pressure, glucose level or oxygen level, or by a signal received from an internalized biologic measurement device.

Several possible indications for the inventive transvascular neuromodulator are depicted in FIGS. 5-14 of the drawings. Conventional neuromodulators are implanted with the stimulation electrode in direct juxtaposition to the target nerve or organ. Limitations include electrode migration, peri-electrode fibrosis, surgical intervention to position the electrode, and possible non-specific energy scatter to stimulate non-target organs.

In contrast, in the invention the electrode(s) are implanted intravascularly, such that the active areas of the electrode(s) are juxtaposed properly to the area being treated.

The invention can be used for at least the following purposes:

Dermatology/Cutaneous:

Thermal injuries

Raynaud's disease

Vasculitis

HEENT:

Retinal spasm

Meniere's disease.

Carotid Stenosis

Cerebrovascular disease

sleep apnea

chronic sinusitis

Pulmonary/Thoracic:

Pulmonary hypertension

Hiccups

Asthma or COPD

Cardiac:

Arrhythmias

post infarction hyperperfusion

angioplasty neovascularity

CABG—revascularization

Gastroenterology:

Esophageal spasm

Gastro-esophageal Reflux Disease (GERD)

Hypomotility

Splanchnic, mesenteric angina

chronic constipation

elimination syndrome

dumping syndrome

Neurology:

Vertebral, subdlavian, and carotid steal syndromes

Post Cerebrovascular Injury

Enhancement of neovascularity

Motion Disorders
Hyperactivity
Hypoactivity
Spasticity
Parkinson's Disease
Huntington's Chorea

Alzheimer's Disease

Multiple Sclerosis

Spinal Cord Injury

Seizures

Urology:

Male and Female Sexual Dysfunction

Urinary Incontinence

Atonic and Neuropathic Bladder

Pelvic Pain Syndrome/Interstitial Cystitis

Ejaculatory dysfunction

Retrograde ejaculation

Anorgasmia

Rheumatology:

Inflammatory syndromes

vasculitis

Endocrinology:

Diabetes: Splenic vein catheterization to elicit pancreatic islet stimulation of autologous Insulin or Glucagon

Thyroid

Adrenal

Vascular:

Neuromodulation of vascular disease

“RF sympathectomy”, central and peripheral

Neovascular collateral stimulation

Renovascular Hypertension

Essential Hypertension

Orthopedic:

bone growth stimulation to treat and prevent osteopenia, osteoporosis

bone deposition for fracture healing

Plastic/Reconstructive Surgery:

improved graft/flap survival

Oncology:

Primary: release of tumor mediators and cytokines

Secondary: Improved vascular supply in conjunction with chemotherapy

Veterinary:

Lactation

Menstrual Cycle regulation

Seminal emission

Several examples are shown in the drawings. The IPG can be operated in “on”, “off” and “pulsatile” modes of operation. In the “on” mode, there is a constant, scheduled course of impulses of designated amplitude, frequency and voltage. In the “off” mode, the IPG is off, in a de-activated state, and activated at a pre-determined time and for a scheduled temporal period by an external source. In the “pulsatile” mode, the IPG is automatically activated by biological regulators or sensors. Examples of biological parameters that can be sensed and used to control the IPG include: temperature, blood flow, oxygen saturation and content, carbon dioxide saturation and content, seizure activity, alterations in electrical activity or muscular activity, distension, pressure, intravascular blood-pressure regulation, and intravascular monitoring of alterations of glucose, electrolytes, pharmacologic agents, vital chemical markers and biological gases such as oxygen, carbon dioxide and ammonia. After these stimuli are sensed as being above or below a predetermined threshold, as appropriate, the IPG is activated. The IPG can then be deactivated after a predetermined time of a predetermined course of impulses, or can be deactivated once the desired clinical threshold or response is accomplished as determined by the monitor.

The inventive trans vascular neuro modulation (TVNM) is an attractive alternative to conventional therapies in the management of chronic medical disease processes. These conditions have hitherto required systemic medical therapies, which are non-specific and delayed in their onset of clinical action. Moreover, such medical therapies have been documented to have significant morbidities and decreased clinical compliance.

TVNM is an evolutionary therapy with increased precision and refinement with electrical stimuli delivered directly to the target organ with minimal, deleterious effects on the adjacent organs or the host itself.

The electrode or electrodes that exit from the IPG can be solitary or multiple (e.g. dual or triple). Solitary electrode leads can be placed in isolated vessels at a precise target organ. Multiple leads can be placed along a large target area or when vessels bifurcate; this allows for a larger organ to be stimulated at multiple focal points. Additionally, one lead could deliver a low voltage, high amplitude for “stimulation” of different cell types, whereas the alternative lead would deliver a high amplitude, low frequency stimulus if a negative response was desired, or a specific response not activated by the previous amplitude and frequency.

FIGS. 5-14 show illustrative, non-limiting examples of use of the inventive TVNM in several treatment modalities, as indicated in the drawings. These also illustrate possible placement of the active electrodes 18 at the distal or active end of electrode member 16, as well as the use of multiple electrode members, such as electrode members 16a and 16b, FIG. 13.

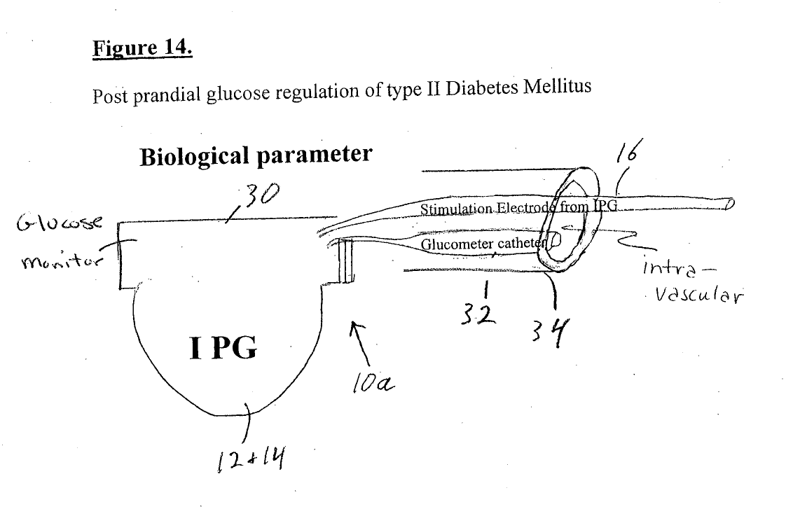

FIG. 14 illustrates in-vivo biological monitoring and control of a monitored parameter, in this example postprandial glucose regulation of type II Diabetes Mellitus. Implanted glucose monitor 30 has glucometer catheter 32 that is implanted in vessel 34; monitor 30 and catheter 32 together sense alterations in blood glucose. The device determines elevated postprandial glucose levels and signals the IPG to generate a course of stimulating impulses through electrode 16 that is located in the splenic vein 34, which elicits endogenous insulin secretion from pancreatic islet cells.

The glucose monitor can additionally detect depressed levels of glucose, signaling the IPG to alter the signal sent through the splenic vein, creating a transvascular stimulation along a predetermined length of the pancreas. This activation stimulates glucagon excretion and suppresses insulin excretion.

Thus, the IPG can be coupled to a biological monitor and regulate life-threatening organ systems using electrical stimulation.

Although specific features of the invention are shown in some drawings and not others, this is for convenience only as the features may be combined in other manners in accordance with the invention.

Other embodiments will occur to those skilled in the art and are within the following claims.

What is claimed is:

1. A method for managing a patient's condition, comprising the steps of:
a. providing an implantable delivery device, comprising:
at least one power source member;
at least one pulse-generating member; and
at least one electrode member having an active portion with one or more electrodes, the electrode member electrically connected to the power source member and/or the pulse generating member, and the electrodes adapted to provide electrical stimulation;
b. surgically implanting the device in the patient such that the power source and pulse generating member are in the body and the active portion of the electrode member is located in one or more blood vessels; and
c. selectively activating the pulse generating member to generate electrical pulses that are provided to one or more of the electrodes, to electrically stimulate the patient's tissue proximate the electrodes.

2. The method of claim 1, wherein the power source member and the pulse-generating member are housed in a biocompatible shell that is adapted to be implanted in the patient.

3. The method of claim 1, wherein the pulse-generating member is adapted to generate pulses of about 10 to about 40 Hz

4. The method of claim 1, wherein the pulse-generating member is adapted to generate pulses of about 1 to about 5.5 V.

5. The method of claim 1, wherein the power source member and the pulse-generating member are adapted to be deactivated automatically after a predetermined temporal period has passed.

6. The method of claim 1, wherein the device further comprises an elongated lead, to which the electrode member is fixed, that connects the electrode member to the power source member and/or the pulse-generating member.

7. The method of claim 1, wherein the power source member comprises a high impedance battery.

8. The method of claim 1, wherein the pulse-generating member emits low amplitude, high frequency pulses.

9. The method of claim 1, wherein the device further comprises a lead with an outside diameter of about 2 mm or less, to which the electrode member is attached and comprises at least one extension cable having a length sufficient to connect the electrode member to the power source member and/or the pulse-generating member.

10. The method of claim 1, wherein the pulse-generating member is deactivated automatically when a predetermined electrical potential is reached.

11. The method of claim 1 wherein the condition is a cutaneous condition.

12. The method of claim 1 wherein the condition is a head, eyes, ears, nose and throat (HEENT) condition.

13. The method of claim 1 wherein the condition is a pulmonary/thoracic condition.

14. The method of claim 1 wherein the condition is a cardiac condition.

15. The method of claim 1 wherein the condition is a gastroenterology condition.

16. The method of claim 1 wherein the condition is a neurology condition.

17. The method of claim 1 wherein the condition is a urology condition.

18. The method of claim 1 wherein the condition is a rheumatology condition.

19. The method of claim 1 wherein the condition is an endocrinology condition.

20. The method of claim 1 wherein the condition is a vascular condition.

21. The method of claim 1 wherein the condition is an orthopedic condition.

22. The method of claim 1 wherein the condition is an oncology condition.

23. The method of claim 1 wherein the patient is non-human.

24. The method of claim 1 wherein the implantable delivery device further comprises an in vivo biological monitor that is electrically coupled to the pulse-generating member.

25. The method of claim 24 wherein the step of selectively activating the pulse generating member is responsive to the biological monitor, such that the pulse-generating member is activated in response to an internal condition sensed by the biological monitor.

26. The method of claim 25 wherein the pulse-generating member is also deactivated in response to an internal condition sensed by the biological monitor.

27. An implantable delivery device for managing a patient's condition, comprising:
a power source member adapted for placement in the body;
a pulse-generating member adapted for placement in the body; and
at least one electrode member having an active portion adapted for intravenous placement in the body and having one or more electrodes, the electrode member electrically connected to the pulse generating member, and the electrodes adapted to provide electrical stimulation to tissue through blood flowing proximate the active portion;
an in vivo biological monitor that senses an internal condition and that is electrically coupled to the pulse-generating member;
wherein the pulse generating member is activated in response to an internal condition sensed by the biological monitor.

28. The device of claim 25 wherein the pulse-generating member is also deactivated in response to an internal condition sensed by the biological monitor.
